(19) United States
(12) Patent Application Publication
(10) Pub. No.: US 2002/0187123 A1
(43) Pub. Date: Dec. 12, 2002

(54) Therapeutic compositions and methods for the inhibition of angiogenesis

(76) Inventors: David Milstone, Chestnut Hill, MA (US); Joyce Bischoff, Weston, MA (US); Ying Yu, Boston, MA (US)

Correspondence Address: Michael A. Sanzo, Fitch, Even, Tabin & Flannery, Suite 401L, 1801 K Street, N.W., Washington, DC, 20006-1201, US

(21) Appl. No.: 10/138,512

(22) Filed: May 6, 2002

Related U.S. Application Data
(60) Provisional application No. 60/288,784, filed on May 7, 2001.

(51) Int. Cl. A61K 38/20; A61K 38/21; A61K 38/22

(52) U.S. Cl. 424/85.2; 424/85.5; 424/85.7; 514/12

(57) ABSTRACT

The present invention is directed to compositions and methods involving the combined use of an angiogenesis inhibitor and an inducer of endogenous E-selectin. Improved methods for treating patients by inhibiting blood vessel growth are described.

### CROSS REFERENCE TO RELATED APPLICATION

The present application claims the benefit of U.S. provisional application No. 60/288,784, filed on May 7, 2001.

### STATEMENT OF GOVERNMENT FUNDING

The United States Government has a paid-up license in this invention and the right in limited circumstances to require the patent owner to license others in reasonable terms as provided for by the terms of NIH Grant Nos. R29-HL54095 and P01-HL36028 awarded by the Department of Health and Human Services.

### FIELD OF THE INVENTION

The present invention is directed to therapeutic compositions and procedures that can be used in the treatment of diseases characterized by abnormal blood vessel growth. In particular, the invention encompasses preparations that can be used to inhibit angiogenesis associated with the growth of solid tumors.

### BACKGROUND OF THE INVENTION

When solid tumors first develop, they obtain nutrients from surrounding tissue by diffusion. However, a point is soon reached at which this process is no longer sufficient to maintain cellular life. In order for the tumor to continue growing, it must attract and become invaginated with blood vessels, a process termed “angiogenesis.” In the 1970's, research was initiated with the objective of developing an anti-cancer agent that would act by blocking angiogenesis. This research led to the discovery of “endostatin,” a polypeptide which has been reported to produce a dramatic reduction in tumor growth when examined in a mouse model (U.S. Pat. No. 6,174,861).

Other research has focused on the ability of cancer cells to enter into a patient's bloodstream and metastasize. In some types of cancer, this process may be mediated, in part, by E-selectin, a cell surface glycoprotein expressed by the endothelium (Ye, et al., Int. J. Cancer 16:455-460 (1995); U.S. Pat. No. 5,081,034)). Studies on the effect of E-selectin on angiogenesis have, for the most part, suggested that this protein promotes the growth of new blood vessels (Nguyen, et al., Nature 365:267-269 (1993); Gerritsen, et al., 75:175-184 (1996); Koch, et al., Nature 376:517-519 (1995)). In addition, at least one reference has suggested that inhibitors of angiogenesis promote the synthesis of E-selectin by endothelial cells (Budson, et al., Biochem. Biophys. Res. Commun. 225:141-145 (1996); Luo, et al., Biochem. Biophys. Res. Commun. 245:906-911 (1998)). Despite these efforts, the exact role of E-selectin in angiogenesis and its relationship to other factors modulating the growth of blood vessels are not understood.

### SUMMARY OF THE INVENTION

The present invention is based upon the discovery that endostatin will not act as an inhibitor of angiogenesis when in vivo levels of E-selectin are deficient. This discovery led the inventors to the concept that low levels of E-selectin can contribute to tumor angiogenesis in certain individuals and that such patients can be treated by the administration of an agent that increases endogenous levels of E-selectin. A second related concept is that the failure of certain patients to respond to endostatin or other inhibitors of angiogenesis may be due to an insufficiency of E-selectin expression. Thus, a combination treatment in which an agent that increases in vivo levels of E-selectin is administered together with an inhibitor of angiogenesis should produce better results than the administration of either agent alone. A third important concept is that agents that inhibit the activity of E-selectin, e.g., activity-inhibiting monoclonal antibodies, may be administered to patients in situations in which one wants to increase, rather than decrease, angiogenesis. This might be desirable, for example, during wound healing or in certain patients with cardiovascular problems or diabetes.

In its first aspect, the invention is directed to a pharmaceutical composition in unit dose form which has as active ingredients both an inhibitor of angiogenesis and an agent that increases endogenous levels of E-selectin. These active ingredients should be present at a concentration sufficient to inhibit angiogenesis when one or more unit doses are administered to a patient. The agent that increases endogenous levels of E-selectin should be distinct from the angiogenesis inhibitor and may be either E-selectin itself or any of the factors described in the art that increase E-selectin levels. Such factors include tumor necrosis factor alpha, interleukin-1 and other cytokines (Bevilacqua, et al., Science 3:1160 (1989); Bevilacqua, et al., Proc. Nat'l Acid Sci. USA 84:9238-9242 (1987)). The term “unit dose” or “unit dosage form” as used herein refers to a single drug administration entity. For example, a single tablet or capsule combining both an inhibitor of angiogenesis and an agent increasing endogenous levels of E-selectin would be a unit dosage form.

Angiogenesis inhibitors that bind heparin constitute a preferred group for use in pharmaceutical compositions with endostatin and angiostatin being most preferred. Other angiogenesis inhibitors that may be used include platelet factor 4, interferon alpha, interferon inducible protein 10, interleukin 12, interferon-gamma, gro-beta, TNP-470, and the 16 kDa N-terminal fragment of prolactin.

The invention includes methods of inhibiting angiogenesis by administering any of the pharmaceutical compositions described above. These compositions should be administered at a therapeutically effective dose, i.e., a sufficient amount should be administered to inhibit the growth of new blood vessels. In cancer patients, this may be reflected by a slowing in the rate at which the cancer spreads or by a shrinkage in tumor mass. In addition to the treatment of patients with solid tumors, other conditions that may be treated include hemangiomas, leukemia, telangietasia, psoriasis, scleroderma, pyogenic granuloma, corneal diseases, rubeosis, neovascular glaucoma, diabetic retinopathy, arthritis, and macular degeneration.

In another aspect, the invention is directed to a method of inhibiting angiogenesis in a patient by administering an inhibitor of angiogenesis and an agent that increases endogenous E-selectin levels. These agents should be administered at a therapeutically effective dose and may be used to treat any of the diseases associated with angiogenesis including all of those recited above. Preferred angiogenesis inhibitors are endostatin or angiostatin and preferred agents for increasing endogenous E-selected levels are either E-selectin itself or a cytokine.

The invention also includes methods of altering blood vessel growth by administering agents that either increase endogenous levels of E-selectin, in cases where the objective is to inhibit angiogenesis, or which inhibit the activity of E-selectin, in cases where the objective is to promote new blood vessel growth. Preferred inhibitors of E-selectin activity are monoclonal antibodies, and E-selectin variants or analogs that have been described in the art (see e.g., U.S. Pat. No. 5,830,871).

### DETAILED DESCRIPTION OF THE INVENTION

The present invention is directed to compositions and methods involving the combination of an inhibitor of angiogenesis and an agent that increases endogenous levels of E-selectin. The term “inhibitor of angiogenesis” as used herein refers to agents other than Eselectin unless otherwise indicated. The most preferred inhibitor is endostatin which is described in U.S. Pat. No. 6,174,861 and which may be obtained using methods disclosed therein. In treatment methods, it should typically be administered to patients at a daily dose of between 0.5 mg/kg and 500 mg/kg, more preferably at a dose of between 1 mg/kg and 100 mg/kg, and most preferably at a dose of between 2 mg/kg and 50 mg/kg. This may be provided in one or more unit dosage forms containing between 15 mg and 1 g of the drug. Other angiogenesis inhibitors may be provided in similar amounts and administered in a similar dosage range. References describing alternative angiogenesis inhibitors and the ways in which they may be obtained and used have been disclosed in the art (see e.g., angiostatin and thrombospondin, Chen et al., Cancer Res. 55:4230-4233 (1995), Good et al., Proc. Nat'l Acid Sci. USA 87:6624-6628 (1990), O'Reilly et al., Cell 79:315-328 (1994); platelet factor 4, Gupta, et al., Proc. Nat'l Acid Sci. USA 92:7799-7803 (1995), Maioni, Science 247:77-79 (1990); interleukin 12 and interferon-gama, Voest, et al., J. Nat'l Cancer Inst. 87:581-586 (1995); gro-beta, Cao, et al., J. Exp. Med. 182:2069-2077 (1995); and the 16 kDa fragment of prolactin, Clapp, et al., Endocrinology 33:1292-1299 (1993)).

E-selectin (also called ELAM-1) is described in U.S. Pat. No. 5,081,034 and methods for obtaining and assaying the protein are also disclosed. In treating patients, E-selectin should typically be administered at a dose of between 1 μg/kg and 10 mg/kg and preferably at a dose of between 1 μg/kg and 1 mg/kg. Thus, the amount present in an individual unit dosage form should typically be between about 25 μg and 2 mg. Several cytokines have been disclosed that also increase endogenous levels of E-selectin and may be administered to patients using prior art references as a guide (see Bevilacqua, et al., Science 3:1160 (1989); Bevilacqua, et al., Proc. Nat'l Acid Sci. USA 84:9238-9242 (1987)).

Any route of administration and type of dosage form is compatible with the present invention provided that it does not result in the destruction of an active ingredient. Routes of delivery may include oral, peroral, internal, pulmonary, rectal, nasal, lingual, transdermal, intravenous, intraarterial, intramuscular, intraperitoneal, intracutaneous and subcutaneous delivery, with parenteral routes being generally preferred. Specific dosage forms may include tablets, pills, capsules, powders, aerosols, suppositories, skin patches, parenterals, and oral liquids. Sustained release formulations may also be used. All dosage forms may be prepared using methods that are standard in the art (see e.g., Remington's Pharmaceutical Sciences, 16th ed. A. Oslo. ed., Easton, Pa. (1980)).

Active ingredients may be used in conjunction with any of the vehicles and excipients commonly employed in pharmaceutical compositions, e.g., talc, gum arabic, lactose, starch, magnesium stearate, cocoa butter, aqueous or non-aqueous solvents, oils, paraffin derivatives, glycols, etc. Coloring and flavoring agents may also be added to preparations designed for oral administration. Solutions can be prepared using water or physiologically compatible organic solvents such as ethanol, 1-2 propylene glycol, polyglycols, dimethyl sulfoxide, fatty alcohols, triglycerides, partial esters of glycerin, and the like. Parenteral compositions containing active ingredients may be prepared using conventional techniques and include sterile isotonic saline, water, 1,3-butanediol, ethanol, 1,2-propylene glycol, polyglycols mixed with water, Ringer's solution, etc.

A patient may be initially given a relatively low dose of a pharmaceutical composition in order to determine whether any adverse side-effects are experienced. This may be particularly important in cases where a patient is taking other medications or has clinical characteristics that suggest that they may not be able to tolerate high drug dosages. Once tolerability has been established, the amount given to the patient may be adjusted upwards as needed. Actual dosages will be determined by the attending physician based upon clinical conditions and using methods well known in the art. Inhibitors of angiogenesis and inducers of E-selectin are, preferably, administered in a single unit dosage form but they may also be given sequentially. The daily dose may be provided as a single bolus or divided into several aliquots.

The results obtained by the inventors indicate that patients that underexpress E-selectin will be less responsive to treatment with an angiogenesis inhibitor. One way to determine in advance if a patient will respond to an inhibitor is to assay a biopsy sample to determine the extent to which E-selectin is expressed in the vicinity of a tumor. If the levels are low relative to those seen in normal individuals, this suggests that treatment using the angiogenesis inhibitor alone is likely to be unsuccessful and that a combination therapy of the type described herein should be employed. Assessment of the success of the treatment method can be made using standard clinical examinations and diagnostic procedures.

In cases where clinical circumstances require that angiogenesis be promoted rather than inhibited, agents that interfere with the activity of E-selectin may be administered. Such agents may take the form of monoclonal antibodies, analogs of E-selectin or derivatives of E-selectin. Dosages may be adjusted using standard methods and procedures may be performed for localized delivery. For example, in order to promote wound healing, E-selectin inhibitors may be applied topically.

### EXAMPLES

A. Corneal Angiogenesis Assay

The effect of endostatin on bFGF-stimulated corneal angiogenesis was examined in wild type and E-selectin deficient mice. These experiments revealed that endostatin delivered by an intraperitoneal osmotic pump inhibited FGF-stimulated angiogenesis in the wild type mice but not in the E-selectin deficient mice.

B. Aortic Ring Explant Assay

The results described above were extended using an aortic ring explant assay of endothelial vessel morphogenesis. The results obtained demonstrated increased rapidity and sensitivity of VEGF-stimulated endothelial vessel formation by aortic endothelium from E-selectin deficient mice compared to wild type mice. Mice aortae contain significant amounts of biologically active endostatin that has been shown to limit endothelial outgrowth in the aorta explant assay. The more rapid and extensive growth in E-selectin deficient aortae is therefore consistent with the hypothesis that E-selectin is required for endostatin activity. Most importantly, vessel formation was inhibited by endostatin only in aortae of wild type mice and not in aortae from E-selectin deficient mice. This confirms the results obtained in the corneal angiogenesis assay, suggests that E-selectin is required in the endothelium itself (or at least in vascular cells) for endostatin activity and makes it unlikely that the in vivo results reflect differences in biodistribution, bioavailability, etc. of endostatin administered to wild type and E-selectin deficient mice.

C. Other Experiments

Other experiments suggest that: a) E-selectin can be sufficient to confer endostatin responsiveness to isolated, cultured human endothelial cells; b) inflammatory mediators that stimulate E-selectin expression are, at least in some instances, able to confer endostatin responsiveness to isolated, cultured human endothelial cells; and c) tumor necrosis factor-alpha appears to block and/or prevent endostatin responsiveness in isolated cultured human endothelial cells even in the presence of E-selectin.

D. Conclusions

The results described above lead to the conclusion that blood vessels performing essential functions allowing tumors to expand beyond the size that can be supported by simple diffusion will respond to endostatin treatment only in the presence of endogenous E-selectin. Tumor blood vessels in patients in which E-selectin expression or function is reduced or eliminated, will not respond. These conclusions apply not only to patients having solid tumors but also to other diseases and conditions in which the inhibition of angiogenesis represents a treatment alternative.

All references cited herein are fully incorporated by reference. Having now fully described the invention, it will be understood by those of skill in the art that the invention may be performed within a wide and equivalent range of conditions, parameters and the like, without affecting the spirit or scope of the invention or any embodiment thereof.

What is claimed is:

1. A pharmaceutical composition in unit dose form comprising as active ingredients:
(a) an inhibitor of angiogenesis; and
(b) an agent that increases endogenous levels of E-selectin;
wherein said active ingredients are present at a concentration sufficient to inhibit angiogenesis when one or more of said unit doses are administered to a patient.

2. The pharmaceutical composition of claim 1, wherein said angiogenesis inhibitor is of the type that binds heparin.

3. The pharmaceutical composition of claim 1, wherein said angiogenesis inhibitor is selected from the group consisting of: endostatin; angiostatin; thrombospondin; platelet factor 4; interferon alpha; interferon inducible protein 10; interleukin 12; interferon-gamma; gro-beta; and the 16 kDa N-terminal fragment of prolactin.

4. The pharmaceutical composition of claim 1, wherein said angiogenesis inhibitor is endostatin or angiostatin.

5. The pharmaceutical composition of any one of claims 1-4, wherein said agent that increases endogenous levels of E-selectin is either a cytokine or E-selectin itself.

6. A method of inhibiting angiogenesis in a patient, comprising: administering to said patient a therapeutically effective dose of the pharmaceutical composition of claim 1.

7. The method of claim 6, wherein said patient has a disease or condition selected from the group consisting of: an hemangioma; a solid tumor; leukemia; telangiectasia; psoriasis; scleroderma; pyogenic granuloma; a corneal disease; rubeosis; neovascular glaucoma; diabetic retinopathy; arthritis; and macular degeneration.

8. The method of claim 7, wherein said patient has a solid tumor.

9. A method of inhibiting angiogenesis in a patient, comprising administering to said patient:
(a) an inhibitor of angiogenesis;
(b) an agent that increases endogenous E-selectin levels in said patient;
wherein said inhibitor of angiogenesis and said agent that increases endogenous levels of E-selectin are administered at a therapeutically effective dosage.

10. The method of claim 9, wherein said angiogenesis inhibitor is of the type that binds heparin.

11. The method of claim 9, wherein said angiogenesis inhibitor is selected from the group consisting of: endostatin; angiostatin; thrombospondin; platelet factor 4; interferon alpha; interferon inducible protein 10; interleukin 12; interferon-gamma; gro-beta; and the 16 kDa N-terminal fragment of prolactin.

12. The method of claim 9, wherein said angiogenesis inhibitor is either angiostatin or endostatin.

13. The method of any one of claims 9-12, wherein said agent that increases endogenous E-selectin levels is either a cytokine or E-selectin itself.

14. The method of claim 9, wherein said patient has a disease or condition selected from the group consisting of: an hemagioma; a solid tumor; leukemia; telangiectasia; psoriasis; scleroderma; pyogenic granuloma; a corneal disease; rubeosis; neovascular glaucoma; diabetic retinopathy; arthritis; and macular degeneration.

15. The method of claim 14, wherein said patient has a solid tumor.

16. A method of treating a patient to prevent the growth or spread of a solid tumor, comprising administering to said patient an agent that increases endogenous E-selectin levels at a dose sufficient to inhibit blood vessel growth.

17. The method of claim 16, wherein said agent that increases endogenous E-selectin levels is a cytokine or E-selectin itself.
